(19) United States
(12) Patent Application Publication
(10) Pub. No.: US 2019/0247077 A1
(43) Pub. Date: Aug. 15, 2019

(54) ULTRASOUND PROBE

(71) Applicant: OLYMPUS CORPORATION, Tokyo (JP)

(72) Inventors: Ken FUJISAKI, Sagamihara-shi (JP); Kiichiro SAWADA, Hachioji-shi (JP); Yasuo TANIGAMI, Hachioji-shi (JP); Hideto YOSHIMINE, Hachioji-shi (JP); Ken YOKOYAMA, Fussa-shi (JP); Ryo MIYASAKA, Hachioji-shi (JP)

(73) Assignee: OLYMPUS CORPORATION, Tokyo (JP)

(21) Appl. No.: 16/391,678

(22) Filed: Apr. 23, 2019

Related U.S. Application Data
(63) Continuation of application No. PCT/JP2016/082183, filed on Oct. 28, 2016.

(51) Int. Cl. A61B 17/32; A61B 17/16

(52) U.S. Cl. CPC A61B 17/320068; A61B 17/1675; A61B 17/320016; A61B 2217/005; A61B 2017/320073

(57) ABSTRACT

An ultrasound probe includes: a probe main body section to which ultrasonic vibration generated by an ultrasound transducer is transmitted; a controller that is connected to the ultrasound transducer and controls generation of the ultrasonic vibration; and a treatment section that is provided on a distal side of the probe main body section and forms a hole in a bone, which is a treatment target, with the ultrasonic vibration. The treatment section has a distal end and a proximal end and includes a columnar portion, a cutting portion that cuts the bone by crushing the bone with the ultrasound vibration and forms the hole, and a discharge portion that discharges cutting debris of the bone cut by the cutting portion toward the proximal end from the cutting portion, the discharge portion including a recessed portion formed in a groove shape inclined with respect to the longitudinal axis.

### CROSS-REFERENCE TO RELATED APPLICATION

This application is a continuation of International Application No. PCT/JP2016/082183, filed on Oct. 28, 2016, the entire contents of which are incorporated herein by reference.

### BACKGROUND

The present disclosure relates to an ultrasound probe.

A typical ultrasound probe can have treatmentsection in a distal portion of the probe with a cutting blade that determines a contour of the hole that is formed in the bone. A plurality of cutting elements of the cutting blade can be radially disposed with respect to a center axis of the probe. debris resulting from cutting the bone can be discharged from a passage formed between the cutting elements on an outer periphery of the treatment section. The passage can be formed as an inclined surface that is separated from the center axis along the treatment section from a distal side toward a proximal side of thereof. Hence, when forming a through-hole in the bone, the treatment section can form a through-hole having a substantially circular contour with substantially the same inner diameter from one end to the other end of the through-hole.

Since the plurality of cutting elements of the cutting blade of the treatment section of the probe are radially disposed with respect to the center axis of the probe, an outer edge approaches or is separated from the center axis when the outer edge of the treatment section of the probe is viewed along a peripheral direction of the center axis. In addition, the outer edge of the treatment section changes in shape toward the proximal side from a distal end. Therefore, when a recessed hole that does not penetrate the bone is to be formed by using the probe, the hole has a specific geometrical contour as it is closer to a back side of the hole.

For example, in a case where a hamstring (semitendinosus muscle) is used in an anterior cruciate ligament reconstructive surgery of a knee joint, a tendon transplant is formed so that a cross section thereof has a rectangular outline or an outline similar thereto. Therefore, even in the case of the through-hole, there are needs of forming a polygonal hole such as a rectangular hole or a hole having an appropriate shape such as an ellipse similar thereto, not a circular hole.

In addition, in a case where a patellar muscle tendon is used, there is a demand that a substantially rectangular parallelepiped-shaped bone on an end portion of the tendon transplant be inserted and fixed into the recessed hole. Therefore, there is a demand that the contour of the bone hole have the same or substantially the same shape on a back side and on an entrance side in a range of the bone hole in which the tendon transplant is inserted into the bone hole, and the bone hole be formed to have a desired simple shape such as a polygon or an ellipse.

### SUMMARY

An ultrasound probe that has a treatment section having a distal end and a proximal end. The treatment section includes a cutting portion that defines a columnar portion. The columnar portion has a projection shape of a polygon, a substantially polygonal shape, an elliptical shape, or a substantially elliptical shape when viewed from the distal end. The probe also has a discharge portion that discharges cutting debris of the bone cut by the cutting portion toward the proximal end from the cutting portion, the discharge portion including a recessed portion with grooves inclined with respect to the longitudinal axis of the treatment section.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic view illustrating a treatment system according to an exemplary embodiment;

FIG. 2 is a schematic view illustrating a treatment unit according to an exemplary embodiment;

FIG. 3A is a schematic view illustrating an ultrasound probe of a treatment instrument of the treatment unit according to an exemplary embodiment;

FIG. 3B is a sectional view illustrating a state in which the ultrasound probe of the treatment instrument of the treatment unit according to an exemplary embodiment is cut along line 3B-3B orthogonal to a longitudinal axis in FIG. 3A;

FIG. 3C is a schematic view illustrating a state of the ultrasound probe of the treatment instrument of the treatment unit according to an exemplary embodiment when viewed from a direction represented by arrow 3C in FIG. 3A;

FIG. 3D is a sectional view illustrating a state in which an ultrasound probe of a treatment instrument of a treatment unit according to an exemplary embodiment is cut along line 3B-3B orthogonal to the longitudinal axis in FIG. 3A;

FIG. 3E is a schematic view illustrating a state of the ultrasound probe of the treatment instrument of the treatment unit according to an exemplary embodiment when viewed from the direction represented by arrow 3C in FIG. 3A;

FIG. 4A is a partial sectional view schematically illustrating a state in which a hole is formed in a bone by the ultrasound probe of the treatment instrument of the treatment unit according to an exemplary embodiment;

FIG. 4B is a prospective view schematically illustrating a recessed hole in the bone, which is formed by the ultrasound probe of the treatment instrument of the treatment unit according to an exemplary embodiment;

FIG. 5A is a schematic view illustrating an ultrasound probe of a treatment instrument of a treatment unit according to an exemplary embodiment;

FIG. 5B is a sectional view illustrating a state in which the ultrasound probe of the treatment instrument of the treatment unit according to an exemplary embodiment is cut along line 5B-5B orthogonal to the longitudinal axis in FIG. 5A;

FIG. 5C is a schematic view illustrating an ultrasound probe of a treatment instrument of a treatment unit according to an exemplary embodiment;

FIG. 5D is a sectional view illustrating a state in which the ultrasound probe of the treatment instrument of the treatment unit according to an exemplary embodiment is cut along line 5D-5D orthogonal to the longitudinal axis in FIG. 5C;

FIG. 6A is a schematic view illustrating a relationship between a columnar portion of a cutting portion and a recessed portion of a discharge portion of a treatment section of the ultrasound probe of the treatment instrument of the treatment unit according to an exemplary embodiment;

FIG. 6B is a schematic view illustrating a state of the columnar portion of the cutting portion and the recessed portion of the discharge portion of the treatment section of the ultrasound probe of the treatment instrument of the treatment unit according to an exemplary embodiment when viewed from a direction represented by arrow 6B in FIG. 6A;

FIG. 7A is a schematic view illustrating a relationship between a columnar portion of a cutting portion and a recessed portion of a discharge portion of a treatment section of an ultrasound probe of a treatment instrument of a treatment unit according to an exemplary embodiment;

FIG. 7B is a schematic view illustrating a state of the columnar portion of the cutting portion and the recessed portion of the discharge portion of the treatment section of the ultrasound probe of the treatment instrument of the treatment unit when viewed from a direction represented by arrow 7B in FIG. 7A;

FIG. 8A is a schematic view illustrating a relationship between a columnar portion of a cutting portion and a recessed portion of a discharge portion of a treatment section of an ultrasound probe of a treatment instrument of a treatment unit;

FIG. 8B is a schematic view illustrating a state of the columnar portion of the cutting portion and the recessed portion of the discharge portion of the treatment section of the ultrasound probe of the treatment instrument of the treatment unit when viewed from a direction represented by arrow 8B in FIG. 8A;

FIG. 9A is a schematic view illustrating a state in which a through-hole is formed from an intercondylar fossa of a femur toward an outer side of the femur by using a drill;

FIG. 9B is a schematic view illustrating a state in which a recessed hole is formed from the intercondylar fossa of the femur toward the outer side of the femur with respect to a drill hole illustrated in FIG. 9A;

FIG. 9C is a schematic view illustrating a state in which an STG-type tendon transplant instead of an anterior cruciate ligament is disposed into the hole illustrated in FIG. 9B, and the tendon transplant is fixed to an outer site of the femur by a fixing instrument;

FIG. 10A is a schematic view illustrating a state in which a through-hole is formed from an intercondylar fossa of a femur toward an outer side of the femur by using a drill;

FIG. 10B is a schematic view illustrating a state in which a longitudinal axis of the cutting portion of the treatment section of the ultrasound probe of the treatment instrument of the treatment unit is disposed at a position displaced from a center axis of the drill hole, and a first recessed hole communicating with the drill hole illustrated in FIG. 10A is formed from the intercondylar fossa of the femur toward the outer side of the femur;

FIG. 10C is a schematic view illustrating a state in which the longitudinal axis of the cutting portion of the treatment section of the ultrasound probe of the treatment instrument of the treatment unit is disposed at a position displaced from the center axis of the drill hole toward an opposite side of the position illustrated in FIG. 10B, and a second recessed hole communicating with the drill hole and the first recessed hole illustrated in FIG. 10B is formed from the intercondylar fossa of the femur toward the outer side of the femur;

FIG. 10D is a schematic view illustrating a state in which a BTB-type tendon transplant instead of the anterior cruciate ligament is disposed into the first recessed hole and the second recessed hole illustrated in FIG. 10C, and the tendon transplant is fixed to the outer site of the femur by the fixing instrument;

FIG. 11A is a schematic view illustrating a state in which the first recessed hole is formed from the intercondylar fossa of the femur toward the outer side of the femur by the cutting portion of the treatment section of the ultrasound probe of the treatment instrument of the treatment unit;

FIG. 11B is a schematic view illustrating a state in which the longitudinal axis of the cutting portion of the treatment section of the ultrasound probe of the treatment instrument of the treatment unit is disposed at a position displaced from the first recessed hole illustrated in FIG. 11A, and the second recessed hole communicating with the first recessed hole illustrated in FIG. 11A is formed from the intercondylar fossa of the femur toward the outer side of the femur; and

FIG. 11C is a schematic view illustrating a state in which the BTB-type tendon transplant instead of the anterior cruciate ligament is disposed into the first recessed hole and the second recessed hole illustrated in FIG. 11B, and the tendon transplant is fixed at a position adjacent to at least one of the first recessed hole and the second recessed hole by a screw.

### DETAILED DESCRIPTION

Embodiments are described with reference to the drawings.

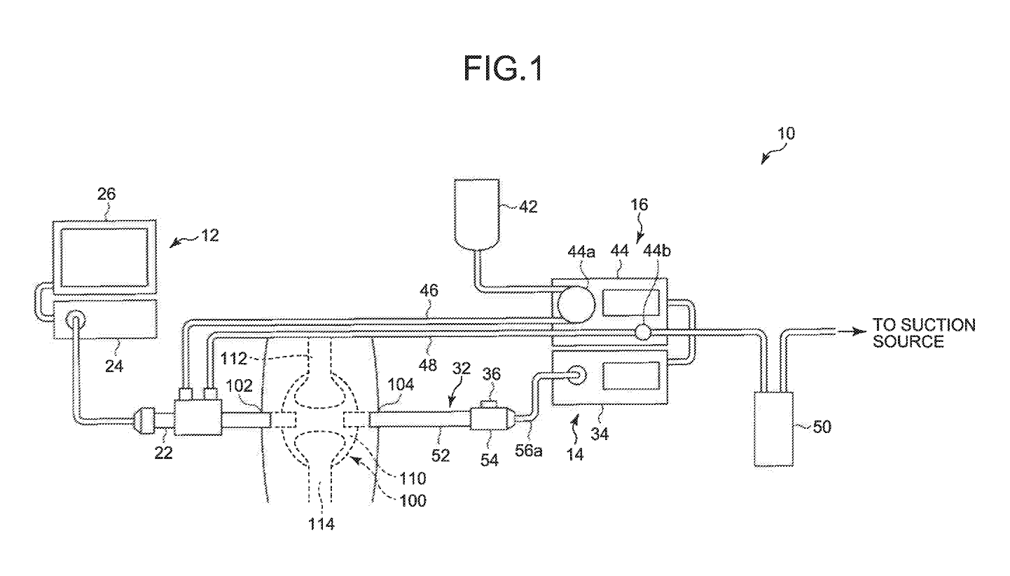

In a case where a treatment is performed on a knee joint 100, a treatment system 10 illustrated in FIG. 1 is used, for example. The treatment system 10 includes an arthroscopic device 12, a treatment device 14, and a perfusion device 16.

The arthroscopic device 12 includes an arthroscope 22 that observes an inside of the knee joint 100, that is, an inside of a joint cavity 110, of a patient, an arthroscope controller 24 that performs an image process based on an object image captured by the arthroscope 22, and a monitor 26 that projects a video generated by the image process by the arthroscope controller 24. The arthroscope 22 is inserted into the joint cavity 110 of the knee joint 100 by a first portal 102 through which the inside of the knee joint 100 and an outside of skin of the patient communicate with each other. A position of the first portal 102 is not fixed but is appropriately determined depending on a condition of the patient. In general, an antero-internal portal and/or antero-external portal is formed as the first portal 102. A cannula (not illustrated) may be provided to the first portal 102, and the arthroscope 22 may be inserted into the joint cavity 110 of the knee joint 100 via the cannula. Incidentally, the arthroscope 22 and a treatment instrument 52 to be described below of the treatment device 14 are delineated in a state in which both are opposite to each other in FIG. 1; however, both are disposed to have an appropriate positional relationship depending on a position or the like of a treatment target.

The treatment device 14 includes a treatment unit 32, a controller 34, and a switch 36. The switch 36 is illustrated as a hand switch in FIG. 1; however, the switch may be a foot switch.

The controller 34 supplies appropriate energy (electric power) to an ultrasound transducer unit 54 to be described below of the treatment unit 32 in response to an operation of the switch 36 and transmits ultrasonic vibration to a treatment section 74 of a probe 66 to be described below of the treatment unit 32. The treatment section 74 of the probe 66 is inserted into the joint cavity 110 of the knee joint 100 by a second portal 104 through which the inside of the joint 100 and the outside of skin of the patient communicate with each other. A position of the second portal 104 is not fixed but is appropriately determined depending on a condition of the patient. Preferably, a cannula (not illustrated) is provided to the second portal 104, and the treatment section 74 of the probe 66 is inserted into the joint cavity 110 of the knee joint 100 via the cannula. For example, the switch 36 maintains a state in which an ultrasound transducer 56b to be described below is driven in a state of a press operation on the switch, and a state in which the ultrasound transducer 56b is driven is canceled when a press on the switch is canceled.

Here, description is provided, in which one switch 36 is used; however, a plurality of switches 36 may be used. The controller 34 is capable of appropriately setting an amplitude of the ultrasound transducer 56b. Therefore, the operation of the switch 36 may change a frequency of ultrasonic vibration which is output from the ultrasound transducer 56b to be described below or may change the amplitude thereof. In addition, the operation of the switch 36 may change both the frequency and the amplitude of ultrasonic vibration which are output from the ultrasound transducer 56b. Hence, preferably, the switch 36 is capable of switching between a plurality of states such as two high and low amplitudes of the ultrasound transducer 56b.

The perfusion device 16 includes a liquid source 42 that contains a perfusate such as a physiological salt solution, a perfusion pump unit 44, a liquid feed tube 46 having one end connected to the liquid source 42, a liquid discharge tube 48, and a suction bottle 50 to which one end of the liquid discharge tube 48 is connected. The suction bottle 50 is connected to the suction source attached to a wall of an operating room. The perfusion pump unit 44 is capable of feeding out the perfusate from the liquid source 42 by a liquid feed pump 44a. In addition, opening and closing of a pinch valve 44b as a liquid discharge valve can cause the perfusion pump unit 44 to switch between suction and suction stop of the perfusate in the joint cavity 110 of the knee joint 100 with respect to the suction bottle 50.

The other end of the liquid feed tube 46 which is a liquid feed tube line is connected to the arthroscope 22. Therefore, it is possible to feed out the perfusate in the joint cavity 110 of the joint 100 via the arthroscope 22. The other end of the liquid discharge tube 48 which is a liquid discharge tube line is connected to the arthroscope 22. Therefore, it is possible to discharge the perfusate from the joint cavity 110 of the joint 100 via the arthroscope 22. Incidentally, it is needless to say that the other end of the liquid discharge tube 48 may be connected to the treatment instrument 52 such that the perfusate can be discharged from the joint 100. Incidentally, the perfusate may be fed and discharged from another portal.

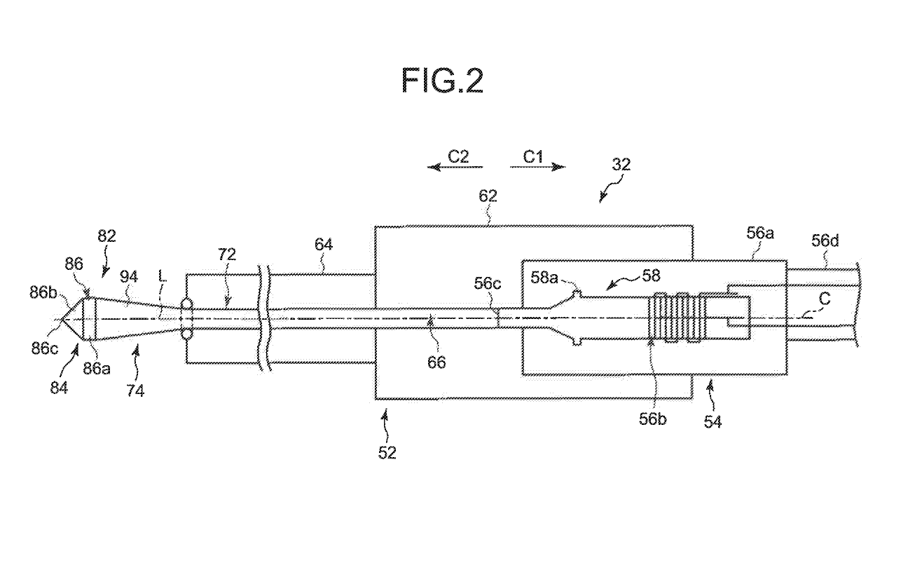

As illustrated in FIG. 2, the treatment unit 32 includes the ultrasound treatment instrument 52 and the ultrasound transducer unit 54. Preferably, the ultrasound transducer unit 54 is attachable to and detachable from the ultrasound treatment instrument 52; however, both may be integrated. The ultrasound transducer unit 54 includes a housing (transducer case) 56a, a bolt-clamped Langevin-type transducer 56b, and a connection portion 56c connected to a proximal end of the ultrasound probe 66 to be described below. The connection portion 56c is formed on a distal end of the transducer 56b. Preferably, the connection portion 56c projects toward a distal side of the housing 56a along a center axis C of the ultrasound transducer unit 54. A cable 56d is extended from a proximal end of the housing 56a of the ultrasound transducer unit 54, the cable having one end that is connected to the transducer 56b and the other end that is connected to the controller 34. The transducer 56b and the connection portion 56c form an integrated vibrating body 58.

The housing 56a supports a support target portion 58a of the vibrating body 58. The ultrasound transducer unit 54 is already known, and thus the detailed description thereof is omitted. In a state in which the transducer 56b generates vibration, the connection portion 56c and a proximal end of the transducer 56b is an antinode of vibration. Incidentally, although not illustrated in FIG. 2, preferably, the switch 36 is provided in the housing 56a of the ultrasound transducer unit 54 or in a housing 62 to be described below of the ultrasound treatment instrument 52.

The ultrasound treatment instrument 52 includes the housing (handle) 62, a cylindrical body (outer cylinder) 64 extended from the housing 62 along the center axis C, and the ultrasound probe 66 inserted into the cylindrical body 64. Here, in the ultrasound treatment instrument 52, a side on which the housing 62 is positioned with respect to the cylindrical body 64 is set to a proximal side (arrow C1 side), and an opposite side of the proximal side is set to a distal side (arrow C2 side). The cylindrical body 64 is attached to the housing 62 from the distal side. In addition, the ultrasound treatment instrument 52 includes the treatment section 74 to be described below at a part on the distal side with respect to the cylindrical body 64.

The housing 62 and the cylindrical body 64 of the ultrasound treatment instrument 52 are formed by a material having an electrical insulation property. The housing 56a of the ultrasound transducer unit 54 is detachably connected to the housing 62 of the ultrasound treatment instrument 52. Preferably, the housing 62 of the ultrasound treatment instrument 52 and the housing 56a of the ultrasound transducer unit 54 are integrated.

Incidentally, a rotating knob (not illustrated) which is a rotation operating member may be attached to the housing 62 of the treatment instrument 52. The rotating knob can rotate around a center axis of the cylindrical body 64 with respect to the housing 62. Rotation of the rotating knob causes the housing 56a of the ultrasound transducer unit 54, the cylindrical body 64, and the treatment section 74 and a probe main body section 72 to be described below to rotate together around the center axis C of the probe main body section 72 with respect to the housing 62.

The housing 62 of the ultrasound treatment instrument 52 and an outer peripheral surface of the cylindrical body 64 have an insulation property. For example, the ultrasound probe 66 is formed by a material by which the ultrasonic vibration can be transmitted, that is, a metal material such as a titanium alloy material. The connection portion 56c of the ultrasound transducer unit 54 fixed to the housing 62 is fixed to the proximal end of the probe 66. Preferably, the entire length of the probe 66 is an integer multiple of a half wavelength based on a resonance frequency of the transducer 56b, for example. The entire length of the probe 66 is not limited to the integer multiple of the half wavelength based on the resonance frequency of the transducer 56b but is appropriately adjusted depending on a material, an amplitude magnification factor, or the like. Therefore, the entire length of the probe 66 may be substantially the integer multiple of the half wavelength based on the resonance frequency of the transducer 56b. Materials or lengths of the vibrating body 58 and the probe 66 are appropriately set such that, as a whole, the vibrating body and the probe vibrate with the resonance frequency of the transducer 56b and a frequency in an output of the controller 34.

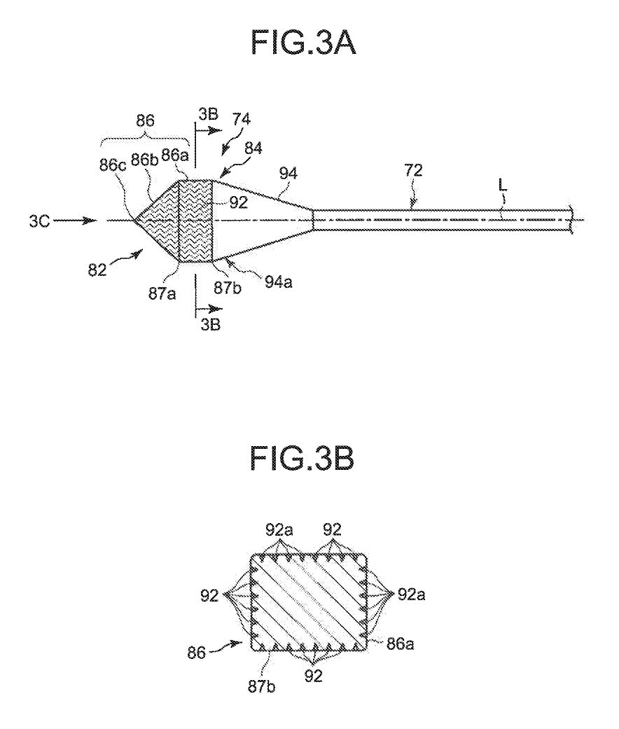

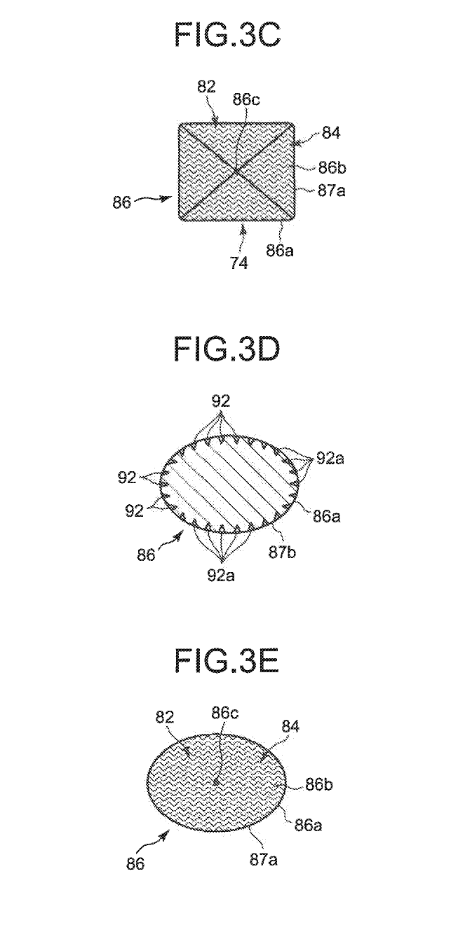

As illustrated in FIGS. 2 and 3A, the ultrasound probe 66 includes the probe main body section 72 and the treatment section 74 that is provided on the distal side of the probe main body section 72 and is capable of forming a hole in a bone which is a treatment target with the ultrasonic vibration. The ultrasonic vibration generated by the ultrasound transducer 56b is transmitted to the probe main body section 72 via the connection portion 56c of the vibrating body 58. The ultrasonic vibration generated by the ultrasound transducer 56b is transmitted to the treatment section 74 via the connection portion 56c and the probe main body section 72.

Preferably, the probe main body section 72 is straightly formed. Preferably, the treatment section 74 is straightly extended from a distal end of the probe main body section 72 toward the distal side; however, the treatment section may be appropriately curved with consideration for visual recognition of the treatment section 74 by the arthroscope 22. Therefore, the center axis C of the probe main body section 72 and a longitudinal axis L of the treatment section 74 may or may not be coincident with each other.

The treatment section 74 includes a cutting portion 82. The cutting portion 82 has a projection shape of a polygon such as a rectangular shape illustrated in FIGS. 3B and 3C when the proximal side of the cutting portion is viewed from the distal side along the longitudinal axis L of the treatment section 74 or a projection shape of an elliptical shape (including a substantially elliptical shape) illustrated in FIGS. 3D and 3E. The projection shape may be a substantially polygonal shape similar to the elliptical shape. The polygon may be a regular polygon. The projection shape may be a rectangle with rounded corners, which is a substantially polygonal shape, a track shape of an athletics stadium, which is a substantially elliptical shape, or the like. Therefore, the projection shape is formed into an appropriate shape such as the polygonal shape, the substantially polygonal shape, an elliptical shape, or a substantially elliptical shape.

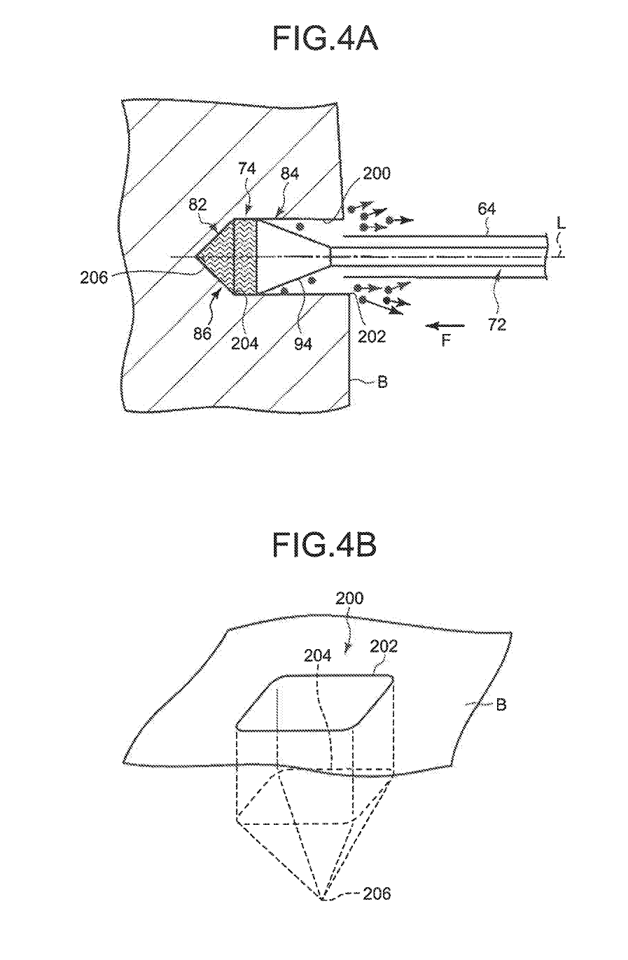

As illustrated in FIG. 4A, the cutting portion 82 of the treatment section 74 is moved such that the treatment section 74 applies a force F to a bone B toward the distal side along the longitudinal axis L, in a state in which the ultrasonic vibration is transmitted to the probe main body section 72. Therefore, the probe 66 is straightly or substantially straightly moved toward the distal side along the center axis C. In this case, the bone is cut by the treatment section 74.

The cutting portion 82 has a block body 86 on a distal portion of the treatment section 74. The block body 86 is formed into a block shape that determines an outline (contour of a hole) when the bone B is cut. The block body 86 is provided with a columnar portion 86a and a projecting portion 86b that projects from the columnar portion 86a toward the distal side along the longitudinal axis L. The columnar portion 86a is formed into a columnar shape such as a polygonal columnar shape or an elliptical columnar shape. The columnar portion 86a and the projecting portion 86b are integrally formed by cutting work or the like.

The columnar portion 86a of the block body 86 of the cutting portion 82 is formed to have a cross section of the same shape or substantially the same shape from a distal end 87a to a proximal end 87b along the longitudinal axis L, the cross section being orthogonal to the longitudinal axis L. An outer peripheral surface of the columnar portion 86a is continuous to the proximal side of the distal end 87a of the columnar portion 86a along the longitudinal axis L. Therefore, the columnar portion 86a is formed to have the cross section with the same area or substantially the same area from the distal end 87a to the proximal end 87b, the cross section being orthogonal to the longitudinal axis L. The distal end 87a of the columnar portion 86a determines the maximum outline portion (contour of the hole) when the bone B is cut. The outer peripheral surface of the columnar portion 86a has the same projection shape as the projection shape of the cutting portion 82 when the proximal side is viewed from the distal side along the longitudinal axis L of the treatment section 74. In this manner, an outline of the cutting portion 82 of the treatment section 74 is formed depending on a shape of the hole (refer to FIG. 4B) which is to be formed when the bone B is cut. Hence, the cutting portion 82 forms a hole in the bone B based on the projection shape.

Incidentally, as a polygonal column of the columnar portion 86a, a triangular column, a quadrangular column, a pentagonal column, a hexagonal column, or the like is formed to have an appropriate shape or a shape similar thereto. The columnar portion 86a does not need to be formed to absolutely have a distinct corner. In addition, the columnar portion 86a does not need to have a regular polygonal shape and is preferably formed in a flat state. Therefore, a use of the probe 66 according to the embodiment enables a hole with a desired shape to be formed in the bone B.

Preferably, the projection shape of the cutting portion 82 is a polygonal shape such as a substantially rectangular shape illustrated in FIGS. 3B and 3C or an elliptical shape illustrated in FIGS. 3D and 3E, for example. In a case where an anterior cruciate ligament reconstructive surgery is performed by using an STG tendon to be described below, an outline of a cross section of a tendon transplant is formed into a substantially rectangular shape having a size of about 4 mm×5 mm, the cross section being orthogonal to the longitudinal axis. Therefore, in a case where the cutting portion 82 has the projection shape of the substantially rectangular shape, as an example, preferably, the outline of the cross section orthogonal to the longitudinal axis L is formed to have a size of about 4 mm×5 mm, for example.

The projecting portion 86b is formed on the distal side of the columnar portion 86a. The projecting portion 86b projects from the distal end 87a of the columnar portion 86a toward the distal side along the longitudinal axis L and is formed to have a cone shape or a substantial cone shape based on the projection shape of the cutting portion 82. A vertex 86c of the projecting portion 86b of the cutting portion 82 is formed at an appropriate position on the distal side along the longitudinal axis L with respect to the columnar portion 86a. The vertex 86c of the projecting portion 86b of the cutting portion 82 is formed within a range of the projection shape of a boundary (distal end 87a of the columnar portion 86a) between the projecting portion 86b and the columnar portion 86a of the cutting portion 82, when the proximal side is viewed from the distal side along the longitudinal axis L. The projecting portion 86b of the cutting portion 82 may be formed to have a straight or curved line formed by connecting the vertex 86c and one point on the boundary between the projecting portion and the columnar portion 86a of the cutting portion 82. Therefore, the shape of the projecting portion 86b of the cutting portion 82 is not limited to the cone shape but may have the substantial cone shape. In addition, the vertex 86c does not need to be formed to have a sharp shape and may have a blunt shape.

Here, the projecting portion 86b of the cutting portion 82 is set to be formed as a quadrangular cone illustrated in FIG. 3C. A contact area between the vertex 86c of the projecting portion 86b of the cutting portion 82 and the bone is small in an initial state when the bone is cut. Therefore, it is possible to start cutting the bone in a state in which friction between the cutting portion 82 and the bone is low.

Here, the vertex 86c of the most distal end of the projecting portion 86b of the cutting portion 82 is moderately sharp. When the vertex 86c abuts or comes into press contact with the bone B with an appropriate force, it is more difficult for the vertex 86c to slide with respect to the bone B, compared with the blunt shape of the vertex. Therefore, when the ultrasonic vibration is transmitted to the probe 66 in a state in which the vertex 86c abuts or comes into press contact with the bone B with the appropriate force, it is difficult for the vertex 86c to slide with respect to the bone B or be displaced when a hole 200 (refer to FIGS. 4A and 4B) starts to be opened. Hence, when the vertex 86c is moderately sharp, it is difficult to shift the position of the vertex 86c of the most distal end of the projecting portion 86b of the cutting portion 82 with respect to the bone B, and it is easy to position a location where the hole 200 is formed.

As illustrated in FIGS. 3A to 3C, the treatment section 74 has a discharge portion 84 that discharges cutting debris of the bone cut by the cutting portion 82 toward the proximal side from the cutting portion 82. A part of the discharge portion 84 is provided in the cutting portion 82. The discharge portion 84 is provided with a recessed portion 92 formed on an outer peripheral surface of the cutting portion 82 and a shaft portion 94 provided on the proximal side from the cutting portion 82.

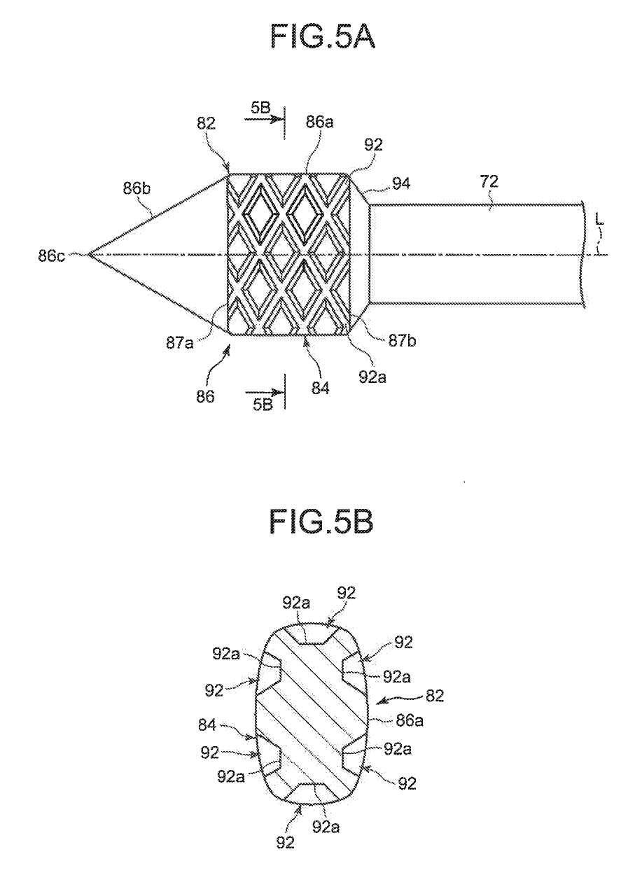

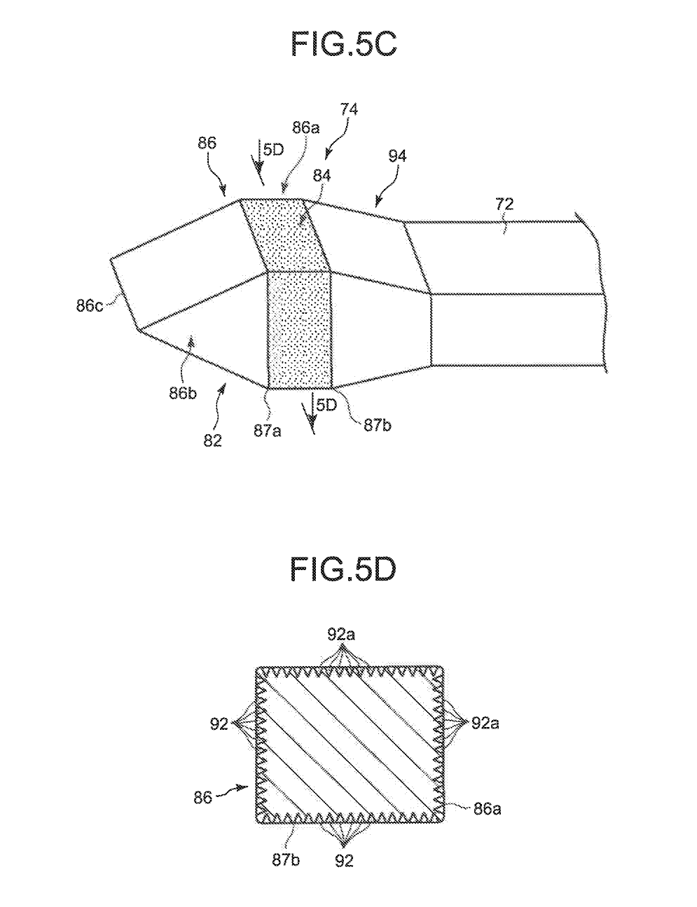

As illustrated in FIG. 3B, the outer peripheral surface of the columnar portion 86a is provided with the recessed portion 92 of the discharge portion 84, which decreases a contact area between the treatment section 74 and the bone and becomes a discharge path of the cutting debris. Here, the recessed portion 92 is formed into a wavy shape with a bottom at a position recessed from outer peripheral surfaces of the columnar portion 86a and the projecting portion 86b. The bottom of the recessed portion 92 is close to the center axis C (longitudinal axis L) than the columnar portion 86a. As will be described below, the recessed portion 92 does not absolutely need to be formed in the projecting portion 86b (refer to FIG. 5A).

The shaft portion 94 is extended more toward the proximal side along the longitudinal axis L than the block body 86 of the cutting portion 82. The shaft portion 94 is provided between the distal end of the probe main body section 72 and the proximal end 87b of the block body 86 of the cutting portion 82. The shaft portion 94 has a projection shape within a range of a projection shape of the block body 86 of the cutting portion 82 when the proximal side is viewed from the distal side along the longitudinal axis L.

The shaft portion 94 is provided with a distal portion 94a that is continuous to the proximal end of the block body 86. The distal portion 94a of the shaft portion 94 has a cross section that decreases in sectional area from the distal side toward the proximal side along the longitudinal axis L, the cross section being orthogonal to the longitudinal axis L. In addition, the shaft portion 94 has a part in which the sectional area of the cross section increases or is maintained to be constant from the distal side toward the proximal side in a part closer to proximal side than the distal portion 94a, the cross section being orthogonal to the longitudinal axis L. In other words, the shaft portion 94 has a narrow portion between a distal end and a proximal end of the shaft portion. A boundary between the distal portion 94a of the shaft portion 94 and the proximal end (proximal end 87b of the columnar portion 86a) of the block body 86 has a shape that prevents stress concentration in a state in which the ultrasonic vibration is transmitted. Therefore, the boundary between the distal portion 94a of the shaft portion 94 and the proximal end 87b of the columnar portion 86a of the block body 86 is smoothly continuous to each other. Incidentally, when the treatment section 74 is viewed from the distal side toward the proximal side along the longitudinal axis L, the shaft portion 94 is hidden by the block body 86, and thus it is not possible to observe the shaft portion 94. Therefore, the shaft portion 94 that is continuous to the proximal side of the block body 86 can be a part of the discharge portion 84 that discharges cutting debris of a bone or a liquid such as a perfusate toward the proximal side along the longitudinal axis L.

When the treatment section 74 is viewed in a direction represented by arrow 3C in FIG. 3A, that is, the proximal side is viewed from the distal side along the longitudinal axis L, an outline of the treatment section 74 is observed as outlines of the projecting portion 86b and the columnar portion 86a of the cutting portion 82, as illustrated in FIG. 3C. In this case, the columnar portion 86a is provided with the recessed portion 92 of the discharge portion 84; however, the outer peripheral surface of the columnar portion 86a appears at least once between the distal end 87a and the proximal end 87b of the columnar portion 86a on an outer edge of the treatment section 74 in FIG. 3C. Therefore, the cutting portion 82 determines the maximum outline portion. Hence, when the proximal side is viewed from the distal side along the longitudinal axis L, the projection shape of the cutting portion 82 becomes a shape of a hole when the bone B is cut by using the treatment instrument 52.

Here, the recessed hole 200 having a desired shape is provided with an opening edge 202 having the same shape and size as those of the projection shape of the cutting portion 82 of the treatment section 74 when the proximal side is viewed from the distal side along the longitudinal axis L, and the recessed portion is straightly recessed toward a back side with the same shape as the shape of the opening edge 202. Therefore, an example of the desired shape of the hole 200 is a rectangular shape having an appropriate depth.

In order to form the recessed hole 200 having the desired shape, the cutting portion 82 of the treatment section 74 needs to have the maximum outline portion such that projection when the proximal side is viewed from the distal side along the longitudinal axis L becomes the shape of the opening edge 202 of the desired hole. The distal end 87a of the columnar portion 86a of the cutting portion 82 of the treatment section 74 is formed into the same shape as the shape of the opening edge 202 of the desired hole 200. Therefore, the distal end 87a of the columnar portion 86a of the cutting portion 82 of the treatment section 74 of the probe 66 of the embodiment allows the recessed hole 200 provided with the desired opening edge 202 to be formed.

On the other hand, from a viewpoint of a decrease in friction between the bone B and the cutting portion 82 of the treatment section 74 and from a viewpoint of discharge of the cutting debris from the bone B, the shorter the length of the maximum outline portion of the cutting portion 82 in a direction (ultrasonic vibration direction) along the longitudinal axis L, the better. Therefore, a configuration in which the columnar portion 86a does not have the same shape and the same sectional area but have a sectional area that gradually decreases from the distal end 87a of the columnar portion 86a, which is the maximum outline portion, toward the proximal side.

Preferably, the probe 66 is straightly moved along the longitudinal axis L, and the hole 200 is straightly formed along the longitudinal axis L by the columnar portion 86a of the cutting portion 82. Therefore, in order to prevent the cutting portion 82 from being unstable and form the hole 200 straightly, the outline of the columnar portion 86a from the distal end 87a toward the proximal end needs to have a certain length by which the columnar portion is parallel to the longitudinal axis L.

In addition, while the ultrasonic vibration having an appropriate amplitude is transmitted to the probe 66, the treatment section 74 cuts the bone B. Therefore, the columnar portion 86a of the cutting portion 82 of the treatment section 74 needs to have an appropriate strength. When the sectional area gradually decreases from the distal end 87a of the columnar portion 86a toward the proximal side, there is a possibility that it is difficult to form the treatment section 74 that has a strength to the extent that the treatment section 74 cuts the bone B, while the ultrasonic vibration having the appropriate amplitude is transmitted to the probe 66, depending on a decrease rate of the sectional area.

The columnar portion 86a of the cutting portion 82 of the probe 66 of the embodiment has a part, which configures the maximum outline portion and is continuous from the distal end 87a to the proximal end 87b, and a certain length along the longitudinal axis L. In the embodiment, the columnar portion 86a of the cutting portion 82 has the cross section that is the same or substantially the same from the distal end 87a to the proximal end 87b of the columnar portion 86a, the cross section being orthogonal to the longitudinal axis L. In this manner, the cutting portion 82 of the treatment section 74 has the columnar portion 86a, and thereby it is possible to the straight hole 200 that has the same shape as the shape of the maximum outline portion of the columnar portion 86a when the bone B is cut, while the strength of the treatment section 74 when the probe 66 is straightly moved toward the distal side along the longitudinal axis L is maintained.

Incidentally, in a case where the columnar portion 86a has the appropriate length in the longitudinal axis L and is not provided with the recessed portion 92 of the discharge portion 84, friction between the bone B and the outer peripheral surface of the columnar portion 86a increases. The columnar portion 86a has the maximum outline portion from the distal end 87a to the proximal end 87b along the longitudinal axis L, and thus outlines of parts orthogonal to the longitudinal axis L are the same at any position from the distal end 87a to the proximal end 87b. Therefore, in a case where the recessed portion 92 of the discharge portion 84 is not present, cutting debris from the bone B cut on the distal end 87a of the columnar portion 86a is pinched between the bone B and the outer peripheral surface of the columnar portion 86a and is unlikely to be discharged.

The recessed portion 92 of the discharge portion 84 of the probe 66 according to the embodiment is formed in the columnar portion 86a. The recessed portion 92 of the discharge portion 84 does not change the projection shape of the maximum outline portion of the columnar portion 86a when the treatment section 74 is viewed from the distal side toward the proximal side along the longitudinal axis L. Further, the recessed portion 92 is continuous from the distal end 87a to the proximal end 87b of the columnar portion 86a. Therefore, once the cutting debris enters the recessed portion 92, the cutting debris moves along the recessed portion 92 toward the proximal side from the treatment section 74, as the probe 66 moves forward along the longitudinal axis L. Hence, the treatment section 74 of the probe 66 according to the embodiment solves problems of friction between the bone B and the cutting portion 82, discharge of the cutting debris cut by the cutting portion 82, and strength of the cutting portion 82.

The distal portion 94a of the shaft portion 94 of the discharge portion 84 has a sectional area that decreases from the distal side toward the proximal side. Hence, the probe 66 is provided with a narrow portion formed in cooperation between the proximal end of the shaft portion 94 and the distal end of the probe main body section 72. Therefore, the shaft portion 94 of the discharge portion 84 of the embodiment can form a space for discharging the cutting debris between an inner wall of the recessed hole 200 of the bone B and the shaft portion 94.

Next, an operation of the treatment system 10 according to the embodiment will be described. Here, mainly regarding an operation of the ultrasound probe 66 of the treatment unit 32, the case of forming the recessed hole 200 in the bone B is described.

The ultrasound transducer unit 54 is attached to the ultrasound treatment instrument 52, and the treatment unit 32 is formed. In this case, the proximal end of the ultrasound probe 66 and the connection portion 56c of the ultrasound transducer unit 54 are connected to each other. Incidentally, here, in order to simplify the description, the center axis C of the probe main body section 72 is coincident with the longitudinal axis L of the treatment section 74.

When the switch 36 is operated, energy is supplied from the controller 34 to the ultrasound transducer 56b of the vibrating body 58 fixed to the proximal end of the ultrasound probe 66, and the ultrasound transducer 56b generates the ultrasonic vibration. Therefore, the ultrasonic vibration is transmitted to the ultrasound probe 66 via the vibrating body 58. The vibration is transmitted from the proximal end of the ultrasound probe 66 toward the distal side. In this case, the connection portion 56c on a distal end of the vibrating body 58 and a proximal end of the vibrating body 58 become an antinode of vibration. One point on the center axis C on an inner side of a support target portion 58a becomes a node of vibration. The proximal end of the ultrasound probe 66, which is connected to the connection portion 56c of the vibrating body 58 becomes the antinode of vibration, and thus the cutting portion 82 of the treatment section 74 becomes the antinode of vibration.

The cutting portion 82 of the treatment section 74 becomes the antinode of vibration, and thus the cutting portion is displaced along the longitudinal axis L at a speed (for example, thousands of m/s) based on a resonance frequency of the transducer 56b. Therefore, when the treatment instrument 52 is moved toward the distal side along the longitudinal axis L (center axis C) such that the treatment section 74 comes into press contact with the bone B in a state in which the vibration is transmitted, a portion of the bone B, with which the treatment section 74 comes into contact, is crushed by an action of the ultrasonic vibration. Hence, as the treatment instrument 52, that is, the probe 66, is moved toward the distal side along the longitudinal axis L (center axis C), the recessed hole 200 is formed in the bone B along the longitudinal axis L of the treatment section 74 of the ultrasound probe 66.

Incidentally, in a case where the bone B is present beneath a cartilage, and the treatment section 74 of the ultrasound probe 66 comes into press contact with the cartilage toward the distal side along the longitudinal axis L, a portion of the cartilage, with which the treatment section 74 comes into contact, is resected, and a recessed hole is formed in the cartilage by the action of the ultrasonic vibration.

The projecting portion 86b and the columnar portion 86a of the treatment section 74 of the ultrasound probe 66 are each provided with the recessed portion 92 of the discharge portion 84. The recessed portion 92 of the discharge portion 84 is formed, and thereby a contact area between the cutting portion 82 and the bone B is smaller than a contact area in a case where the recessed portion 92 is not formed, when the recessed hole 200 is formed in the bone B. Therefore, friction between the cutting portion 82 and the bone B is reduced. In addition, the recessed portion 92 is present in the cutting portion 82, and thereby a surface area is larger than that in a case where the recessed portion 92 is not formed. Since a joint fluid or a perfusate is present in the joint 100, heat dissipation capacity of the treatment section 74 improves, and the treatment section is smoothly cooled due to the presence of the recessed portion 92. Hence, cutting debris of the bone B is placed in the recessed portion 92. Therefore, the treatment section 74 of the treatment unit 32 can form the recessed hole 200 at an appropriate speed.

Hence, when the treatment section 74 is viewed from the distal side toward the proximal side along the longitudinal axis L, it is not possible to observe the shaft portion 94 of the discharge portion 84 due to the presence of the columnar portion 86a of the cutting portion 82. Therefore, when the recessed hole 200 is formed, a space is formed between the proximal end 87b of the columnar portion 86a, the shaft portion 94, and a side of the bone hole 200. Therefore, the cutting debris of the bone B is discharged to the space between the shaft portion 94 and the side of the bone hole 200 from the proximal end 87b of the columnar portion 86a.

In this manner, the cutting debris of a site of the bone B, on which a treatment is performed by the treatment section 74, is discharged toward the proximal side through the recessed portion 92 of the discharge portion 84 along the longitudinal axis L. In particular, the joint 100 is filled with the joint fluid. In addition, the perfusate circulates in the joint 100. Therefore, the joint fluid or the perfusate acts as a lubricant such that it is easy for the cutting debris of the bone B to be discharged toward the proximal side from the cutting portion 82 along the longitudinal axis L. In a case where forming of the recessed hole 200 having a desired depth in the bone B is completed, a press on the switch 36 is canceled such that the generation of the ultrasonic vibration is stopped. Hence, the ultrasound probe 66 is moved toward the proximal side along the longitudinal axis L.

As illustrated in FIG. 4B, the recessed hole 200 formed in the bone B is formed into the same shape as an outer edge of the columnar portion 86a of the cutting portion 82 from the entrance 202 to a back-side site 204. A deepest position 206 of the recessed hole 200 is formed into the same shape as an outline of the projecting portion 86b including the vertex 86c. In other words, as illustrated in FIG. 4A, in a case where the ultrasonic vibration is transmitted to the probe 66 of the ultrasound treatment instrument 52 such that the recessed hole 200 is formed in the bone B, it is possible to copy the shape of the cutting portion 82 of the treatment section 74 as it is.

The ultrasonic vibration is transmitted to the probe 66 of the treatment unit 32 according to the embodiment, and the ultrasonic vibration is applied to a site of the bone B, to which a hole needs to be formed. In this manner, the site, with which the treatment section 74 on the distal end of the probe 66 comes into contact, is finely crushed and cut. The distal portion of the treatment section 74 has a projecting shape, and the cutting portion 82 is further provided with the recessed portion 92 of the discharge portion 84 through which the cutting debris of the bone B is discharged. Therefore, the projecting portion 86b or the recessed portion 92 of the discharge portion 84 is provided rather than the cutting portion 82 that is not provided with the projecting portion 86b and has the same shape of the projection shape of the columnar portion 86a in an axial direction without change, and thereby opening work of opening a hole more rapidly can progress.

The cutting portion 82 is moved along the longitudinal axis L, and thereby the shape of the distal end 87a of the columnar portion 86a as it is can be formed as the opening edge of the recessed hole 200 when the treatment section 74 is viewed from the distal side along the longitudinal axis L. Therefore, the projection shape of the cutting portion 82 along the longitudinal axis L is the same as the shape of the desired recessed hole 200. Hence, the cutting portion 82 digs a hole in the bone B, and thereby it is possible to open the recessed hole 200 having a desired shape with a desired depth in the bone B.

In addition, the treatment section 74 has a projecting shape on the distal portion, and the contact area between the bone B and the cutting portion 82 is decreased by the recessed portion 92 of the discharge portion 84. In this manner, it is further easy to discharge the cutting debris. Therefore, when the bone B is cut, it is possible to limit generation of friction between the treatment section 74 and the bone B, and it is possible to increase a work speed.

FIGS. 5A and 5B illustrate a first modification example of the ultrasound probe 66.

As illustrated in FIG. 5B, the cross section of the columnar portion 86a of the treatment section 74 of the ultrasound probe 66 has a substantially elliptical shape, the cross section being orthogonal to the longitudinal axis L. Incidentally, the columnar portion 86a has the same shape between the distal end 87a and the proximal end 87b and has the projection shape of the substantially elliptical shape when the proximal side is viewed from the distal side along the longitudinal axis L of the treatment section 74.

Here, the discharge portion 84 is formed in the columnar portion 86a of the cutting portion 82 of the treatment section 74. On the other hand, the discharge portion 84 is formed in the projecting portion 86b of the cutting portion 82 of the treatment section 74.

In this case, the cutting debris of the cartilage or the bone B cut by the projecting portion 86b of the cutting portion 82 is placed between the projecting portion 86b and the cartilage or the bone B. The cutting debris is moved toward the columnar portion 86a from an inclined surface of the projecting portion 86b, as the recessed hole 200 is formed deeply. Hence, the cutting debris is discharged toward the proximal side along the longitudinal axis L from the recessed portion 92 of the discharge portion 84 between the distal end 87a and the proximal end 87b of the columnar portion 86a. In this case, a discharge amount of the cutting debris from the projecting portion 86b toward the proximal side is more decreased, compared to a case where the discharge portion 84 is present in the projecting portion 86b. Incidentally, an amount of the cutting debris is adjusted by a shape or the like of the recessed portion 92.

The projecting portion 86b of the cutting portion 82 of the ultrasound probe 66 illustrated in FIG. 5A can more decrease the cutting speed, but it is possible to cut the bone B, compared to the projecting portion 86b of the cutting portion 82 of the ultrasound probe 66 illustrated in FIG. 3A. Therefore, the recessed portion 92 of the discharge portion 84 does not absolutely need to be formed in the projecting portion 86b. When the recessed portion 92 of the discharge portion 84 is formed in the projecting portion 86b of the cutting portion 82, the discharge of the cutting debris is more promoted, and the work speed can be increased. After the columnar portion 86a of the cutting portion 82 reaches the bone B, the cutting debris is discharged toward the proximal side along the longitudinal axis L by the recessed portion 92 of the discharge portion 84 which is formed in the columnar portion 86a.

As illustrated in FIG. 5B, a groove (bottom) 92a having a cross-hatched shape is formed, as the recessed portion 92 of the discharge portion 84 in the columnar portion 86a of the cutting portion 82 of the treatment section 74. The groove 92a is continuous from the distal end 87a to the proximal end 87b of the columnar portion 86a. The recessed portion 92 of the discharge portion 84 is provided in the columnar portion 86a; however, the outer edge of the columnar portion 86a does not change as illustrated in FIG. 5B when the treatment section 74 is viewed from the distal side toward the proximal side along the longitudinal axis L. Therefore, the distal end 87a of the columnar portion 86a of the cutting portion 82 can form the recessed hole 200 without a change in outline (contour).

Incidentally, as described above, the groove 92a is continuous from the distal end 87a toward the proximal end 87b of the columnar portion 86a. Therefore, since the cutting debris of the bone B, which enters the groove 92a once, moves along the groove 92a that is continuous from the distal end 87a and the proximal end 87b, and thus the cutting debris is easily discharged from the distal end 87a through the proximal end 87b of the columnar portion 86a toward the proximal side of the treatment section 74.

FIGS. 5C and 5D illustrate a second modification example of the ultrasound probe 66.

The vertex 86c of the ultrasound probe 66 illustrated in FIG. 5C has an edge extended in a direction orthogonal to the longitudinal axis L. The vertex (edge) 86c is parallel to the distal end 87a of the columnar portion 86a. In other words, the projecting portion 86b is not limited to the cone shape. The projecting portion 86b of the cutting portion 82 has a shape in which a sectional area of a cross section orthogonal to the longitudinal axis L decreases from the distal end 87a of the columnar portion 86a of the cutting portion 82 toward the distal side along the longitudinal axis L.

As illustrated in FIGS. 5C and 5D, the recessed portion 92 of the discharge portion 84 is formed in the columnar portion 86a. The recessed portion 92 is provided with the recessed bottom 92a resulted from a sandblast process. Here, as illustrated in FIG. 5D, the projection shape of the treatment section is a rectangular shape when the proximal side is viewed from the distal side along the longitudinal axis L of the treatment section 74. The distal end 87a of the columnar portion 86a determines the maximum outline portion when the bone B is cut, and a cross section of the columnar portion has the same shape or substantially the same shape as the projection shape of the rectangular shape from the distal end 87a to the proximal end 87b along the longitudinal axis L, the cross section being orthogonal to the longitudinal axis L. Hence, when the proximal side is viewed from the distal side of the treatment section 74 along the longitudinal axis L, a proximal end of the recessed portion 92 cannot be visually recognized and is entirely hidden by the columnar portion 86a.

The recessed portion 92 processed by sandblast is provided with an enormous number of bottoms 92a. An enormous number of vertices are formed by the enormous number of bottoms 92a in the maximum outline portion between the distal end 87a and the proximal end 87b of the columnar portion 86a of the cutting portion 82.

Incidentally, the bottom 92a is continuous from the distal end 87a to the proximal end 87b of the columnar portion 86a. An enormous inter-vertex distances of the maximum outline portion of the columnar portion 86a of the cutting portion 82 are formed to be larger than the cutting debris of the bone B. Therefore, the cutting debris enters a space between the enormous number of vertices of the maximum outline portions of the columnar portion 86a of the cutting portion 82. Hence, the cutting debris of the bone B, which enters the bottom 92a once, is easily discharged on the proximal side of the treatment section 74 from the distal end 87a to the proximal end 87b of the columnar portion 86a.

Otherwise, the recessed portion 92 of the discharge portion 84 illustrated in FIG. 5C is preferably a bottom 92a having a recessed spiral groove shape (refer to FIGS. 3B and 3D), a bottom 92a having a recessed cross-hatched shape (refer to FIG. 5B), or the like.

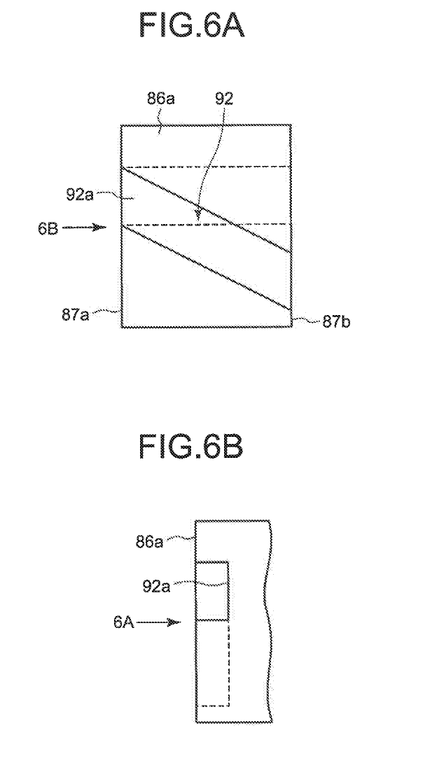

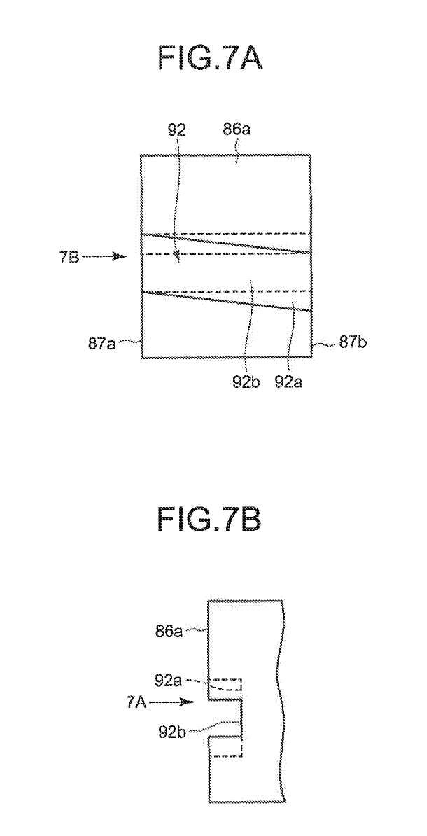

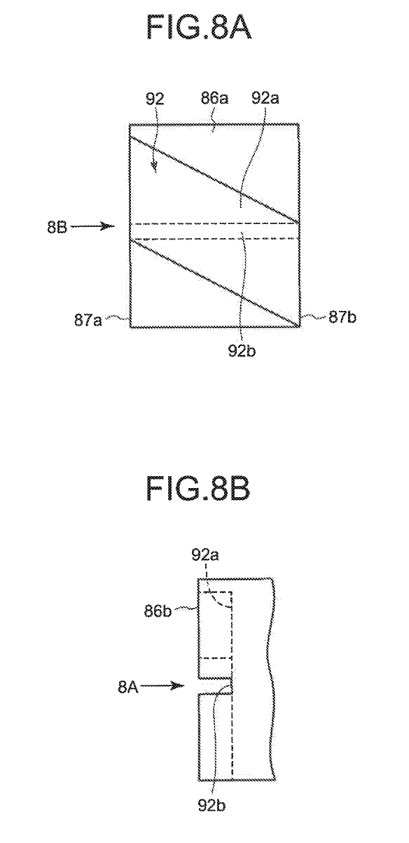

Here, FIGS. 6A and 6B illustrate an example of the recessed portion 92 of the discharge portion 84 formed in the columnar portion 86a. Here, drawing of the projecting portion 86b is omitted. FIGS. 7A and 7B illustrate a reference example of an undesirable recessed portion 92 of the discharge portion 84 formed in the columnar portion 86a. Here, drawing of the projecting portion 86b is omitted. FIGS. 8A and 8B illustrate another reference example of an undesirable recessed portion 92 of the discharge portion 84 formed in the columnar portion 86a. Here, drawing of the projecting portion 86b is omitted.

As illustrated in FIGS. 6A and 6B, the recessed portion 92 of the discharge portion 84 from the distal end 87a to the proximal end 87b of the columnar portion 86a of the cutting portion 82 is inclined such that a proximal end of the bottom 92a of the recessed portion 92 is not observed when the proximal side is viewed from the distal side of the treatment section 74 along the longitudinal axis L. In a case where a part of the proximal end of the bottom 92a of the recessed portion 92 is observed as in the reference example illustrated in FIGS. 7A and 7B, the bone is likely to be uncut straightly along the longitudinal axis L by a groove 92b along the longitudinal axis L.

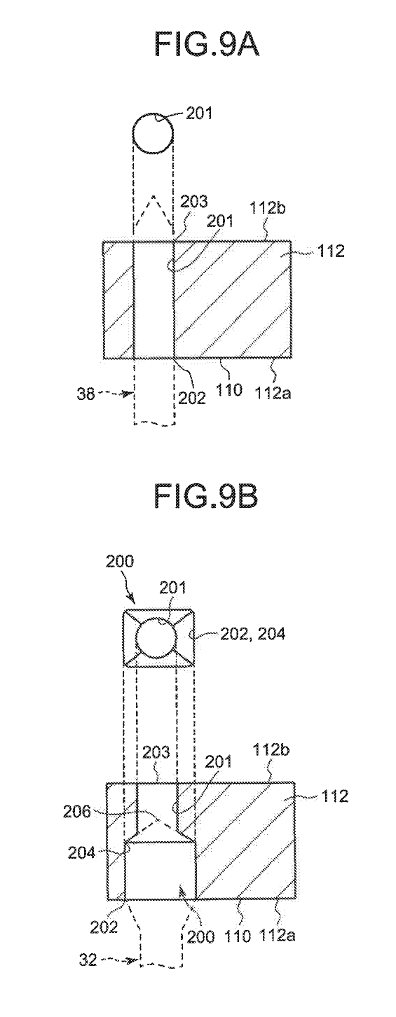

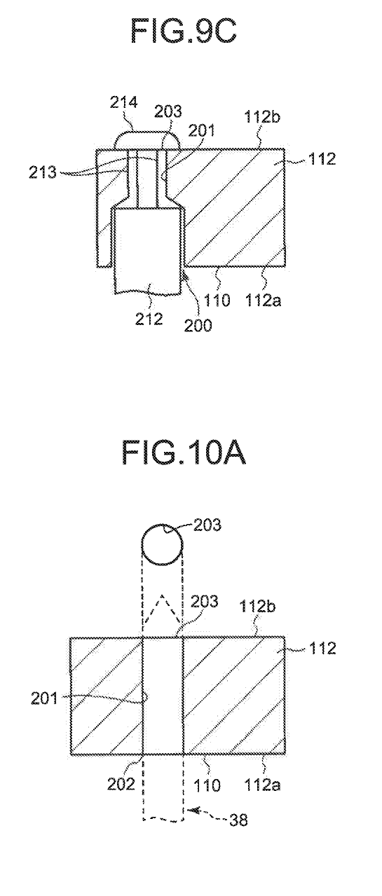

In the reference example illustrated in FIG. 8A, the recessed portion 92 has the same inclination as that in FIG. 6A. However, a width of the recessed portion 92 is larger than that in FIG. 6A, the width being orthogonal to the longitudinal axis L. Hence, when the proximal side is viewed from the distal side of the treatment section 74 along the longitudinal axis L, a part of the proximal end of the bottom 92a of the recessed portion 92 is observed. In this case, as illustrated in FIG. 8B, the bone has a portion that does not abut the columnar portion 86a, from the distal end 87a to the proximal end 87b of the columnar portion 86a. Therefore, an uncut portion of the bone is straightly formed along the longitudinal axis L. There is a concern that a tendon transplant 212 or 216 (refer to FIGS. 9C, 10D, and 11C) to be described below is caught on the uncut portion. In this manner, it is not preferable that the recessed portion 92 have a shape illustrated in FIGS. 8A and 8B. Therefore, the projection shape of the maximum outline portion is not broken, and an angle and a width of the recessed portion 92 is formed as illustrated in FIG. 6A. In addition, only one recessed portion 92 illustrated in FIG. 6A is not formed, but a plurality of recessed portions are preferably formed.

Here, there is provided the simple description of an operation example performed on a side of a femur 112 in a case where the anterior cruciate ligament reconstructive surgery in the knee joint 100 is performed.

It is possible to divide an operation method into two methods depending on a material of a tendon transplant of a ligament to be reconstructed. One is a method in which a semitendinosus muscle tendon or a gracilis muscle tendon present inside a knee is used as the tendon transplant (STG tendon) 212. The other is a method in which a patellar tendon is used as the tendon transplant (BTB tendon) 216. Incidentally, here, in both the methods, the bone hole 200 is formed by an inside-out method from an inside of the joint cavity 110 toward an outer side of the femur 112.

First, an example of using the tendon transplant (STG tendon) 212 is simply described with reference to FIGS. 9A to 9C.

By appropriately using the treatment system 10 illustrated in FIG. 1, the semitendinosus muscle tendon or the gracilis tendon present inside the knee is collected as the tendon transplant 212. In this case, a length of the tendon is substantially about 250 mm to 300 mm. Hence, the collected tendon is bent a plurality of times such as four times to six times, and thereby the tendon transplant 212 having an outline of a cross section of a substantially rectangular shape is formed, the cross section being orthogonal to the longitudinal axis, for example. In this case, an outline of the tendon transplant 212 is 4 mm×5 mm, as an example. Hence, a string 213 illustrated in FIG. 9C is caused to pass to one end of the tendon transplant 212, and a suspended fixing instrument 214 is fixed to the string 213.

A treatment instrument (not illustrated) is put into an inner side of the joint cavity 110 of the knee joint 100 through the second portal (skin incision site) 104 of the knee joint 100. Then, while a footprint of the anterior cruciate ligament on a side of the femur 112 is checked by using the arthroscope 22, the cut anterior cruciate ligament is dissected to expose the footprint (portion to which the anterior cruciate ligament is attached). Incidentally, although not illustrated clearly, the footprint is present in a rear part of an outer wall of an intercondylar fossa of the femur 112. In addition, the footprint on a side of a tibia 114 is present on an inner side of an anterior intercondylar area of the tibia 114. A position of the footprint on the side of the femur 112 is a position, at which one end of a bone hole (tunnel) 201 on the side of the femur 112 is placed, or the vicinity of the position. Here, an example of forming the bone hole 201 and the recessed hole 200 having a desired shape in the footprint is described.

As illustrated in FIG. 9A, a drill 38 is inserted into the joint cavity 110 through the second portal 104 of the knee joint 100. While the footprint of the anterior cruciate ligament on the side of the femur 112 is checked by using the arthroscope 22, the penetrating bone hole (tunnel) 201 that has an end portion in the footprint and is used for the tendon transplant 212 to pass through the femur 112 is opened by using the drill 38. In other words, the bone hole 201 is formed from a site 112a of the femur 112 in the joint 100 to an outer site 112b of the femur 112. In this case, the through-hole 201 has a circular shape.

Then, the drill 38 is removed from the inside of the joint 100, and the treatment section 74 of the probe 66 of the ultrasound treatment instrument 52 is inserted from the same second portal 104, for example. Hence, a state in which the projecting portion 86b of the cutting portion 82 of the treatment section 74 abuts the entrance 202 of the bone hole 201 is checked by using the arthroscope 22. Hence, the ultrasonic vibration is generated by the transducer 56b such that the treatment section 74 is moved forward along the longitudinal axis L. Therefore, as illustrated in FIG. 9B, the recessed hole 200 having a substantially rectangular parallelepiped shape with an appropriate depth is formed in the site 112a of the femur 112 in the joint 100. In this case, the drill hole 201 by the drill 38 and the recessed hole 200 communicate with each other.

As illustrated in FIG. 9C, the tendon transplant 212 is placed in the recessed hole 200. In this case, the recessed hole 200 has a rectangular shape of about 4 mm×5 mm, and the tendon transplant 212 has a rectangular shape of about 4 mm×5 mm. Therefore, the tendon transplant 212 is prevented from rotating around a longitudinal axis of the recessed hole 200 and the drill hole 201. Hence, the fixing instrument 214 is supported by the outer site 112b of the femur 112 through the drill hole 201. In this manner, the tendon transplant 212 is fixed on the side of the femur 112.

Incidentally, since the anterior cruciate ligament is anatomically known as two fiber bundles, it is preferable that two holes be opened at each of the femur 112 and the tibia 114, and the tendon transplants 212 pass through the holes, respectively.

Then, the other end of the tendon transplant 212 is fixed in the vicinity of a front surface of the tibia 114, with a string (not illustrated) passing through the bone hole (not illustrated) formed in the tibia 114 (refer to FIG. 1).

Next, an example of using the tendon transplant (BTB tendon) 216 will be simply described.

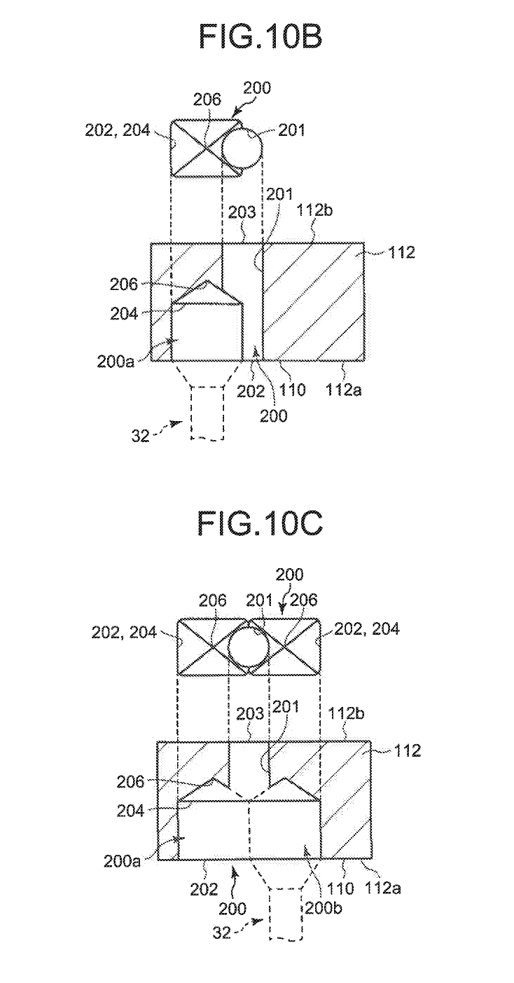

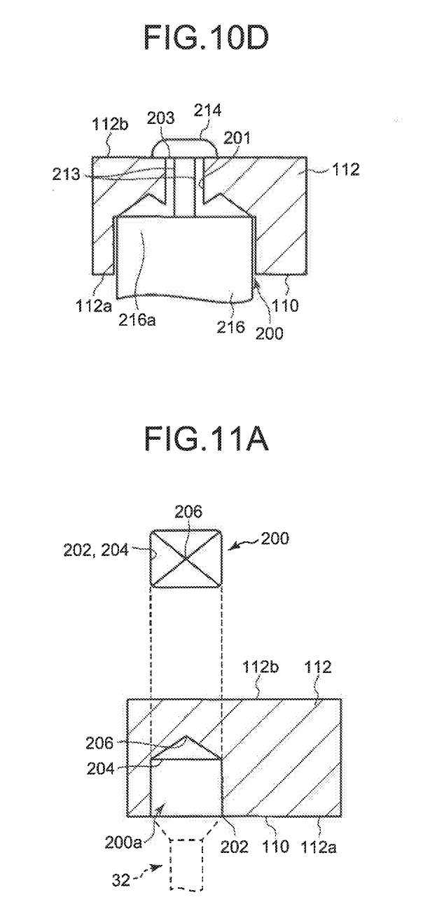

A first example of using the BTB-type tendon transplant 216 is described with reference to FIGS. 10A to 10D.

By using the treatment system 10, the patellar tendon is collected as the tendon transplant 216. In this case, an outline of a bone piece 216a of the tendon transplant 216 is 10 mm×5 mm, as an example. Hence, the string 213 illustrated in FIG. 10D is caused to pass through the tendon transplant 216, and the suspended fixing instrument 214 is fixed to the string 213.

As illustrated in FIG. 10A, similarly to the description provided above, the bone hole (drill hole) 201 is formed in the footprint by using the drill 38. The bone hole 201 is formed from the site 112a of the femur 112 in the joint 100 to the outer site 112b of the femur 112. In this case, the through-hole 201 has a circular shape.

Then, the drill 38 is removed from the inside of the joint 100, and the treatment section 74 of the probe 66 of the ultrasound treatment instrument 52 is inserted from the same second portal 104. Hence, a state in which the projecting portion 86b of the cutting portion 82 of the treatment section 74 abuts the entrance 202 of the bone hole 201 is checked by using the arthroscope 22. Hence, the ultrasonic vibration is generated by the transducer 56b such that the treatment section 74 is moved forward along the longitudinal axis L. Therefore, as illustrated in FIG. 10B, a first recessed hole 200a having an appropriate depth is formed. In this case, the first recessed hole 200a communicates with the drill hole 201 by the drill 38. In this case, the first recessed hole 200a having a substantially rectangular parallelepiped shape is formed in the site 112a of the femur 112 in the joint 100.

The treatment section 74 of the same probe 66 is moved back along the longitudinal axis L such that the treatment section 74 is removed from the first recessed hole 200a. Hence, the position of the treatment section 74 is shifted to a position that is continuously adjacent to the first recessed hole 200a such that the treatment section 74 abuts the bone B. The ultrasonic vibration is generated by the transducer 56b such that the treatment section 74 is moved forward along the longitudinal axis L. Therefore, as illustrated in FIG. 10C, a second recessed hole 200b having an appropriate depth is formed. In this case, the second recessed hole 200b communicates with the drill hole 201 by the drill 38. In this case, the first recessed hole 200a and the second recessed hole 200b which have a substantially rectangular parallelepiped shape are formed in the site 112a of the femur 112 in the joint 100. Hence, the first recessed hole 200a and the second recessed hole 200b are continuous to each other as illustrated in FIG. 10C and both form one continuous recessed hole 200.

Incidentally, the bone piece 216a of the tendon transplant 216 has a rectangular shape of about 10 mm×5 mm. In a case where the recessed hole 200 does not reach the size of the bone piece and the bone piece 216a cannot enter the recessed hole 200, the recessed hole 200 is more widened by using the ultrasound treatment instrument 52.

As illustrated in FIG. 10D, the bone piece 216a of the tendon transplant 216 is placed in the recessed hole 200. In this case, the bone piece 216a of the tendon transplant 216 has a rectangular shape of about 10 mm×5 mm. The recessed hole 200 is formed to have a rectangular shape that is slightly larger than the bone piece 216a of the tendon transplant 216. Therefore, the recessed hole 200 prevents he tendon transplant 216 from rotating around the longitudinal axis of the recessed hole. Hence, the fixing instrument 214 is supported by the outer site 112b of the femur 112. In this manner, the tendon transplant 216 is fixed on the side of the femur 112.

Then, the other end of the tendon transplant 216 is fixed in the vicinity of the front surface of the tibia 114, with a string (not illustrated) passing through the bone hole (not illustrated) formed in the tibia 114 (refer to FIG. 1). Alternatively, the other end of the tendon transplant 216 is fixed by using a screw (not illustrated).

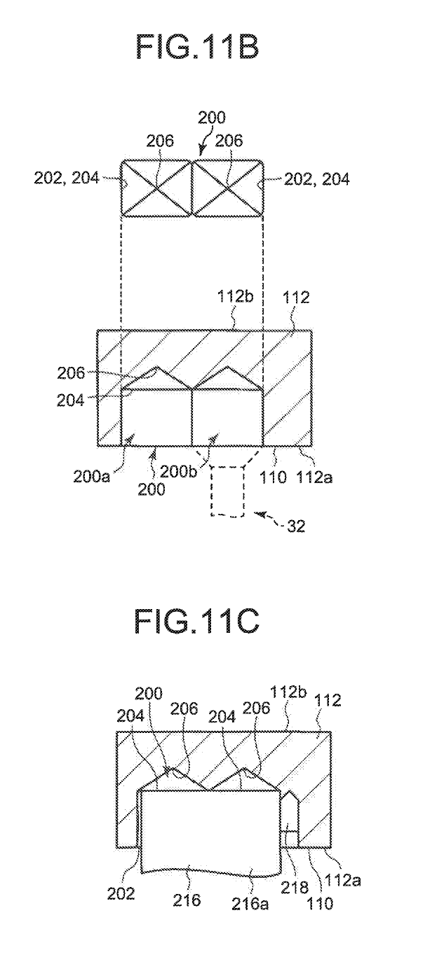

A second example of using the BTB-type tendon transplant 216 is described with reference to FIGS. 11A to 11C.

Here, an example of using a screw 218 as a fixing instrument is described.

As illustrated in FIG. 11A, the treatment section 74 of the probe 66 of the ultrasound treatment instrument 52 is inserted from the second portal 104. Hence, a state in which the projecting portion 86b of the cutting portion 82 of the treatment section 74 abuts the footprint is checked by using the arthroscope 22. In this manner, the ultrasonic vibration is generated by the transducer 56b such that the treatment section 74 is moved forward along the longitudinal axis L. In this case, the first recessed hole 200a having the substantially rectangular parallelepiped shape with an appropriate depth is formed in the site 112a of the femur 112 in the joint 100.

Hence, the position of the treatment section 74 of the same probe 66 is shifted such that the projecting portion 86b of the cutting portion 82 of the treatment section 74 abuts the entrance 202 of the bone hole 201. The ultrasonic vibration is generated by the transducer 56b such that the treatment section 74 is moved forward along the longitudinal axis L. Therefore, as illustrated in FIG. 11B, the second recessed hole 200b having the appropriate depth is formed. The first recessed hole 200a and the second recessed hole 200b which have the substantially rectangular parallelepiped shape are formed in the site 112a of the femur 112 in the joint 100. Hence, the first recessed hole 200a and the second recessed hole 200b are continuous to each other as illustrated in FIG. 11B and both form one continuous recessed hole 200.

As illustrated in FIG. 11C, the bone piece 216a of the tendon transplant 216 is placed in the recessed hole 200. In this case, the recessed hole 200 prevents he tendon transplant 216 from rotating around the longitudinal axis of the recessed hole. In addition, the screw 218 presses the bone piece 216a of the tendon transplant 216 to a wall surface of the recessed hole 200.

In this manner, the tendon transplant 216 is fixed on the side of the femur 112.

As described above, the probe 66 of the treatment instrument 52 according to the embodiment is described as follows.

The footprint of the anterior cruciate ligament takes a narrow area. On the other hand, an outline of an end portion of the tendon transplants 212 and 216 has the rectangular shape or the substantially rectangular shape different from a circle. For example, when the BTB-type tendon transplant 216 of 5 mm×10 mm=50 mm² enters a circular hole formed by a drill, the circular hole needs to have a diameter of about 11 mm. In this case, a sectional area of the circular hole is about 95 mm², and substantially a half of the sectional area is a space. The joint fluid can enter the space, and ligamentation of the tendon transplant 216 can be delayed. The same is true of the case of the STG-type tendon transplant 212.

Therefore, when the recessed hole 200 is formed according to the outline of the tendon transplant 212 or 216, it is possible to reduce a space quantity between the recessed hole 200 and the tendon transplant 212 or 216 and reduce a cutting amount of the bone B. In the embodiment, it is possible to form the recessed hole 200 by using the ultrasound treatment instrument 52 including the treatment section 74 provided with the columnar portion 86a having a cross section of the rectangular shape, the substantially rectangular shape, the elliptical shape, or the substantially elliptical shape. In other words, it is possible to form the recessed hole 200 having the same shape or substantially the same shape as the outline of the tendon transplant 212 or 216 which is buried in the bone B. Therefore, it is possible to form the bone hole 200, in which the end portion of the tendon transplant 212 or 216 is placed without projecting as much as possible with respect to the footprint of the anterior cruciate ligament. Therefore, the tendon transplant is prevented from invading into peripheral tissue of the footprint of the anterior cruciate ligament of the site 112a of the femur 112 in the joint 100. In addition, it is possible to make the ligamentation of the tendon transplants 212 and 216 earlier by the recessed hole 200 having a space quantity to the smallest extent in a state in which the tendon transplant 212 or 216 is placed.

In addition, a treatment of forming the recessed hole 200 in the bone B by the ultrasound treatment instrument 52 is different from a treatment of dilating a hole by a dilator or the like. Therefore, it is possible to perform the treatment on a target of medical treatment, even when the target is a woman, an aged person, or the like who has low bone density and is excluded from the operation target in related art.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound probe comprising:
a probe main body section that receives ultrasonic vibration generated from an ultrasound transducer;
a controller that is connected to the ultrasound transducer and is configured to control generation of the ultrasonic vibration; and
a treatment section that is provided on a distal side of the probe main body section and is configured to form a hole in a treatment target, with the ultrasonic vibration, the treatment section having a distal end and a proximal end and including:
a cutting portion that is configured to cut the treatment target to form the hole while the cutting portion is moved in a direction along a longitudinal axis of the treatment section in a state in which the ultrasonic vibration is transmitted to the probe main body section, the cutting portion including:
a columnar portion having a distal end and a proximal end, and when viewed from the distal end of the treatment section, the columnar portion has a projection shape that is one of the following: a polygon shape; a substantially polygonal shape; an elliptical shape; and a substantially elliptical shape.

2. The ultrasound probe according to claim 1, wherein the treatment section further comprises a discharge portion that is configured to discharge cutting debris of the treatment target cut by the cutting portion toward the proximal end of the treatment section from the cutting portion, the discharge portion including a recessed portion formed in a groove shape, the recessed portion being inclined with respect to the longitudinal axis of the treatment section.

3. The ultrasound probe according to claim 2, wherein a proximal end of the recessed portion is not visible when the proximal end of the treatment section is viewed from the distal end of the treatment section along the longitudinal axis.

4. The ultrasound probe according to claim 2, wherein the recessed portion is provided with a recessed spiral groove-shaped bottom.

5. The ultrasound probe according to claim 2, wherein the recessed portion is provided with a recessed crosshatch-shaped bottom.

6. The ultrasound probe according to claim 1, wherein a cross-section of the columnar portion has a shape that is the same as the projection shape of the columnar portion when viewed from the distal end of the treatment section along the longitudinal axis, the cross-section of the columnar portion being orthogonal to the longitudinal axis.

7. The ultrasound probe according to claim 6, wherein the distal end of the columnar portion defines a maximum outline of a cut section of the treatment target.

8. The ultrasound probe according to claim 2, wherein:
the cutting portion further includes a projecting portion that projects distally from the columnar portion toward along the longitudinal axis; and
the recessed portion of the discharge portion is configured to decrease a contact area between the treatment section and the treatment target such that the recessed portion is a discharge path of the cutting debris.

9. The ultrasound probe according to claim 1, wherein:
the cutting portion further includes a projecting portion that projects distally from the columnar portion along the longitudinal axis; and
the projecting portion of the cutting portion has a cross-sectional area that decreases from the distal end of the columnar portion of the cutting portion toward a distal end of the projecting portion.

10. The ultrasound probe according to claim 2, further comprising a shaft portion that abuts the discharge portion, the shaft portion being provided between the probe main body section and the columnar portion and extends proximally from the columnar portion along the longitudinal axis, the shaft having a dimension that is covered by the projection shape of the cutting portion when viewed from a distal side along the longitudinal axis.

11. The ultrasound probe according to claim 10, wherein:
the shaft portion is provided with a distal portion that is continuous to a proximal end of the columnar portion along the longitudinal axis; and
the distal portion of the shaft portion has a cross section that decreases from a distal side of the shaft portion toward a proximal side of the shaft portion along the longitudinal axis, the cross section being orthogonal to the longitudinal axis.

12. The ultrasound probe according to claim 11, wherein a boundary between the distal portion of the shaft portion and the proximal end of the columnar portion has a shape that prevents stress concentration in a state in which the ultrasonic vibration is transmitted.

13. The ultrasound probe according to claim 1, wherein the columnar portion has a predetermined length from the distal end of the columnar portion to the proximal end of the columnar portion.

14. The ultrasound probe according to claim 1, wherein the columnar portion is a block shape.
